(19) United States
(12) Patent Application Publication
(10) Pub. No.: US 2011/0263901 A1
(43) Pub. Date: Oct. 27, 2011

(54) Process of Preparation of Proguanil

(76) Inventors: Dhananjay Govind Sathe, Mumbai (IN); Harish Kashinath Mondkar, Mumbai (IN); Tanaji Shamrao Jadhav, Mumbai (IN); Nitin Nivrutti Hagavane, Mumbai (IN)

(21) Appl. No.: 12/525,587

(22) PCT Filed: Jan. 13, 2009
(86) PCT No.: PCT/IN2009/000044

§ 371 Date: Sep. 30, 2009

(30) Foreign Application Priority Data
Jan. 23, 2008 (IN) 159/MUM/2008

(51) Int. Cl. C07C 279/26; C07C 277/06; C07C 277/08

(52) U.S. Cl. 564/234

(57) ABSTRACT

Disclosed herein is the process for the preparation of 1-(4-chlorophenyl)-5-isopropyl-biguanide hydrochloride (Proguanil hydrochloride), Formula-I, an antimalarial agent.

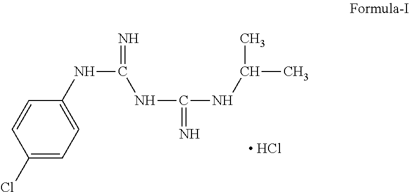

This application claims priority from Indian Application No. 159/MUM/2008 filed on 23 Jan. 2008 and PCT Application No. PCT/IN2009/000044 filed on 13 Jan. 2009.

### TECHNICAL FIELD

The present invention relates to the process for the preparation of 1-(4-chlorophenyl)-5-isopropyl-biguanide hydrochloride (Proguanil hydrochloride), Formula-I, an antimalarial agent.

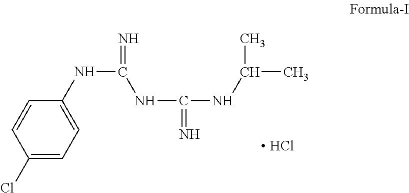

### BACKGROUND AND PRIOR ART

Proguanil (PALUDRINE) is the common name for chloroguanide, a biguanide derivative that emerged in 1945 as a product of British antimalarial drug research. The antimalarial activity of Proguanil eventually was ascribed to cycloguanil, a cyclic triazine metabolite and selective inhibitor of the bifunctional plasmodial dihydrofolate reductase-thymidylate synthetase. Indeed, investigation of compounds bearing a structural resemblance to cycloguanil resulted in the development of antimalarial dihydrofolate reductase inhibitors such as pyrimethamine. Accrued evidence also indicates that Proguanil itself has intrinsic antimalarial activity independent of its effect on parasite dihydrofolate reductase-thymidylate synthetase (Fidock and Wellems, 1997).

Constitution and antimalarial activities of biguanide derivatives are well described in literature. It was found that a wide range of mono and dialkyl derivatives corresponding to Formula-I were active, highest activity occurring with a total of 3 or 4 carbon atoms in the alkyl groups with a maximum at mono-isopropyl. The parent para-chlorophenyl and its monomethyl derivative were inactive. An important feature of these compounds was that their biological activity was not confined to the erythrocytic forms of the malarial parasite (Ann. Trop. Med. Parasite. 1945, 39, 208) but many of these substances act on parasitic phase preceding the blood invasive form, that is, in experimental species at least they function as true casual prophylactic agents.

Proguanil is marketed by Zeneca under the brand name “Paludrine” and in combination with Atovaquone is commercially available under the name Malarone by Glaxo Wellcome. Proguanil hydrochloride prevents malaria by stopping the parasite from reproducing once it is inside the red blood cells. It does this by blocking the action of a compound that is found in the Plasmodium parasite. This compound is an enzyme called dihydrofolate reductase and is involved in the reproduction of the parasite. Dihydrofolate reductase normally converts folic acid into folinic acid in the parasite, which is a step essential for the parasite to produce new genetic material (DNA). New DNA is necessary for the parasite to reproduce. By blocking its production, Proguanil prevents any malarial parasites that have entered the red blood cells from reproducing, increasing in number and causing malaria attacks.

Proguanil hydrochloride, a biguanide derivative, chemically named as 1-(4-chlorophenyl)-5-isopropyl-biguanide hydrochloride is one of the safest antimalarial drugs. This class of compounds has effective antimalarial activity and hence can be used as an antimalarial agent in treatment of human malaria. The relation between antimalarial activity and the chemical constitution of the pyrimidine compounds is well known. Working hypothesis has led to the synthesis and discovery of antimalarial activity of biguanide derivatives as disclosed in J. Chem. Soc., 1946, 729-737. Proguanil hydrochloride is usually taken in combination with another antimalarial medicine called chloroquine to prevent malaria. Various other combinations of Proguanil hydrochloride along with other antimalarial agents like Atovaquone are reported in patents/patent application WO 94/12164, U.S. Pat. No. 6,291,488 and U.S. Pat. No. 3,674,872. In some formulations, Proguanil is combined with antibiotics like X 14868, LL-C23024 beta, LL-C23024 iota (U.S. Pat. No. 4,496,549). Several biguanide derivatives have been examined in human malaria. But ‘Paludrine’ has proved highly effective at doses lower than those customary with mepacrine and quinine (J. Chem. Soc., 1946, 729-737).

J. Chem. Soc., 1946, 729-737 discloses synthesis of Proguanil acetate by reacting p-chlorophenylcyanoguanidine with isopropylamine in presence of copper sulfate pentahydrate in aqueous ethanol. (Scheme-1). After completion of the reaction, sodium sulfide solution is added to the obtained copper complex of Proguanil in hydrochloric acid followed by isolation of the product from mother liquor as free base by treatment with sodium hydroxide. In order to get highly pure material two to three purifications are required which consumes more amount of solvent, thereby making the process economically unviable. Major disadvantage of this process is use of sodium sulfide, to break the formed Proguanil copper complex in the reaction which leads to evolution of hydrogen sulfide (a fatal gas) which needs specially designed facility to perform the process on commercial scale which involves extra investment. Moreover, filtration and workup of copper sulfide after completion of the reaction is very tedious. Thus this process is not ecofriendly and industrially feasible.

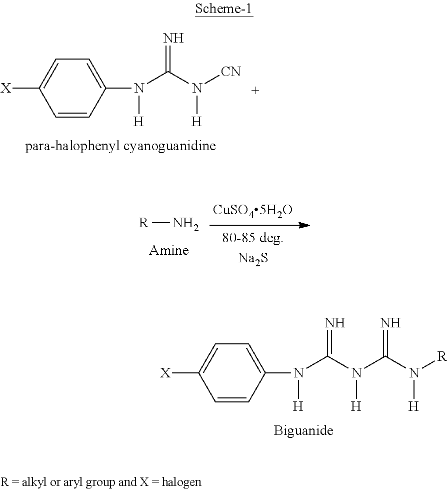

U.S. Pat. No. 2,531,404 and U.S. Pat. No. 2,510,081 discloses preparation of the Proguanil and its salts by reacting p-chlorophenyldicyanamide with isopropylamine in presence of copper sulfate pentahydrate in ethanol, represented by Scheme-1. After completion of the reaction copper complex of Proguanil was dissolved in hydrochloric acid and copper was removed as copper sulfide by addition of sodium sulfide solution and the product was isolated from mother liquor as free base by treatment with sodium hydroxide which can be further converted into its various salts.

U.S. Pat. No. 2,467,371 discloses preparation of Proguanil base followed by its conversion to acetate salt in example 31 by reaction of p-chlorophenyl dicyandiamide with isopropylamine in ethanol in presence of copper sulphate pentahydrate in water. The yield and purity of the product obtained in not reported in this patent. J. Chem. Soc., 1948, 1630-1636 describes synthesis of Proguanil acetate by reacting isopropylcyano guanidine with p-chloroaniline hydrochloride. The disadvantage of this process is that it uses p-chloroaniline which can cause cyanosis hence not suitable for commercial scale.

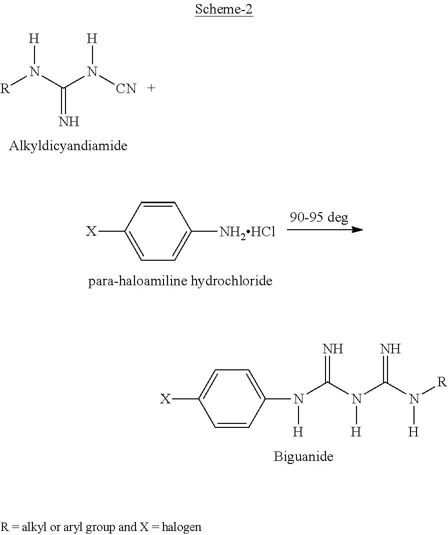

J. Chem. Soc., 1949, 98-106 describes synthesis of Proguanil nitrate by reacting p-chlorophenylguanidine nitrate with isopropylcyanamide. (Scheme-3). In particular, even though literature by Curd, et al, teaches the synthesis of biguanide derivatives but this reference contains no disclosure of the problems which arise in the synthesis of Proguanil hydrochloride and no disclosure of the critical properties of the solvent. Thus, there are no disclosures in the literature which describe the difficulties which arise in synthesizing Proguanil hydrochloride. If the reagents and solvents used in synthesizing Proguanil hydrochloride are not carefully chosen, the reaction may proceed very slowly or not at all and it may be difficult to isolate the desired product.

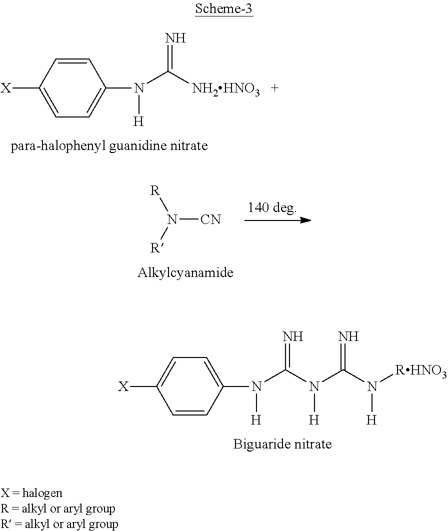

The drawbacks of the above processes for synthesis of the Proguanil hydrochloride as mentioned in prior art are as follows:
a) Use of ethanol-water as solvent system is not suitable to get highest purity material as it provides product with only 80-85% purity.
b) It requires two to three solvent purification, which involves more solvent consumption, to get highly pure material in good yield thereby making the process economically unviable.
c) Use of sodium sulfide for breaking the copper complex formed during the reaction leads to high evolution of a fatal hydrogen sulfide gas thus process is not ecofriendly.
d) It requires 20 hrs or more to complete the reaction which makes the process time consuming.
e) Use of p-chloroaniline which is not safe on commercial scale since p-chloroaniline causes cyanosis after exposure and also it is fatal.

The process of the present invention has several advantages which are as follows:
1. Use of THF-water solvent system in the reaction provides Proguanil hydrochloride with 98 to 99.9% purity (by HPLC).
2. It provides Proguanil hydrochloride in higher yield of 75-90% and with high purity of 99.9% thus the process is cost effective.
3. Evolution of fatal hydrogen sulfide gas is minimized by using less quantity of copper sulfate.
4. This process involves the use of safe reagents such as ethylene diamine tetraacetic acid disodium salt (EDTANa₂) and nitrilotriacetic acid in order to remove copper. Thus evolution of fatal hydrogen sulfide gas is totally avoided during the process.
5. Proguanil hydrochloride prepared by the present invention is obtained in high yield, superior in quality and meeting ICH standards.
6. It requires only 2-10 hours to complete the reaction thus the process is easy and not time consuming.
7. The process is ecofriendly, economically and industrially viable.
8. This process provides the pure product according to strict international standards for active pharmaceutical ingredient with a good quality management.

Thus there is need to develop process for preparation of Proguanil hydrochloride which is simple, safe, ecofriendly, robust and commercially as well as economically feasible. The present invention provides process for producing Proguanil hydrochloride, an antimalarial drug, in high yield and purity.

### OBJECT OF THE INVENTION

The main object of the present invention is to provide the process for the preparation of 1-(4-chlorophenyl)-5-isopropyl-biguanide hydrochloride (Proguanil hydrochloride) of Formula-I in high purity and yield.

Another object of the present invention is to provide a simple, cost effective, ecofriendly and industrially feasible process which is safe to use on commercial scale by avoiding use of unsafe reagent like p-chloroaniline and evolution of most hazardous and fatal hydrogen sulfide gas.

### SUMMARY OF THE INVENTION

The present invention discloses a process for the preparation of 1-(4-chlorophenyl)-5-isopropyl-biguanide hydrochloride (Proguanil hydrochloride) of Formula-I in higher yield and purity.

According to the present invention, there is provided a process for preparation of the 1-(4-chlorophenyl)-5-isopropyl-biguanide hydrochloride (Proguanil hydrochloride) comprising the steps of:
a) reacting p-chlorophenylcyanoguanidine (IV) with molar excess of isopropylamine (V) in suitable solvent in presence of metal salt;

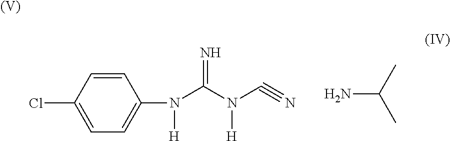

b) adding acid to the obtained Proguanil metal complex;
c) adding chelating agent to the obtained reaction mass;
d) isolating Proguanil hydrochloride;
e) optionally purifying Proguanil hydrochloride to get pure compound of Formula I.

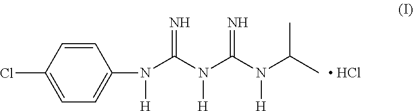

According to another aspect of the present invention there is provided process for purification of Proguanil hydrochloride comprising the step of,
a) dissolving Proguanil hydrochloride in suitable solvent;
b) adding anti-solvent to the obtained solution to get pure Proguanil hydrochloride with purity more than 99%.

The present invention thus provides safe, cost effective, economical and industrially feasible process by avoiding unsafe reagents for the preparation of 1-(4-chlorophenyl)-5-isopropyl-biguanide hydrochloride (Proguanil hydrochloride).

### BRIEF DESCRIPTION OF THE DRAWING

FIG. 1 illustrates X-ray powder diffraction pattern of Proguanil hydrochloride.

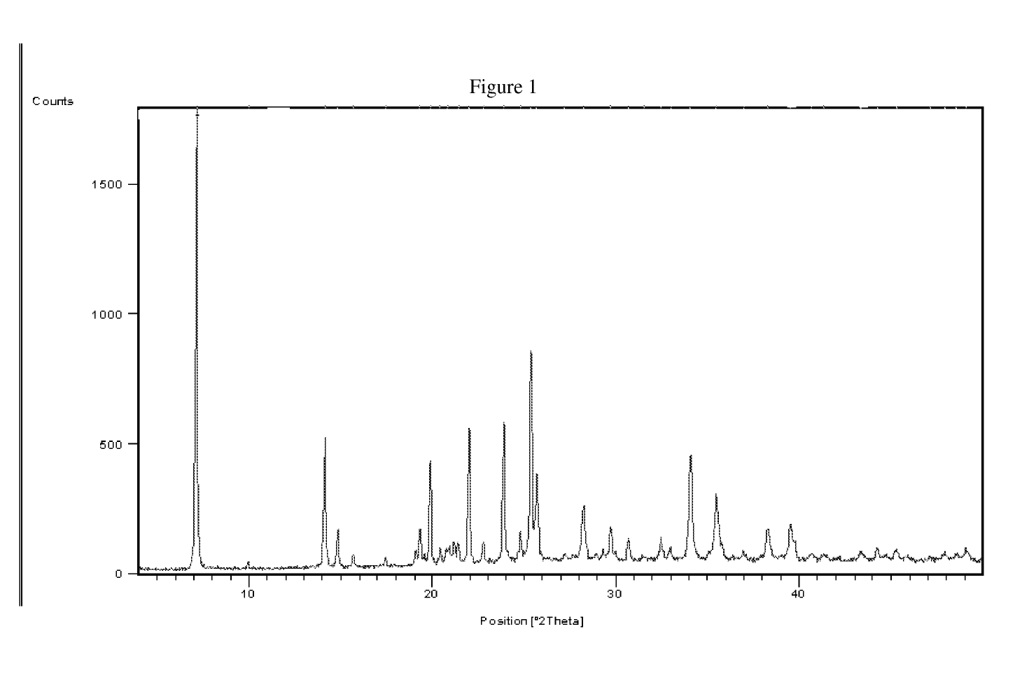

### DETAILED DESCRIPTION OF THE INVENTION

The present invention describes an efficient process for the preparation of 1-(4-chlorophenyl)-5-isopropyl-biguanide hydrochloride (Proguanil hydrochloride), Formula-I in high yield and purity. The process sequence of the present invention is represented by the following scheme,

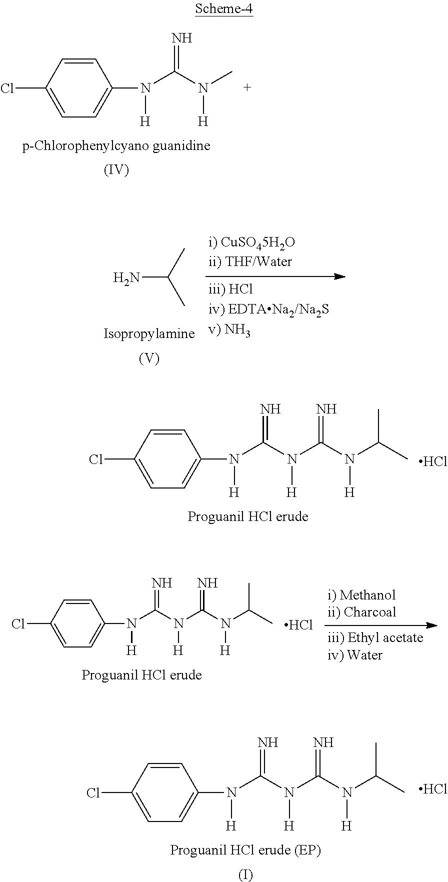

According to the present invention, the process for preparation of 1-(4-chlorophenyl)-5-isopropyl-biguanide hydrochloride (Proguanil hydrochloride) comprises reacting p-chlorophenylcyanoguanidine (IV) with molar excess of isopropylamine (V) to get Proguanil hydrochloride.

According to another embodiment of the present invention there is provided a process for preparation of 1-(4-chlorophenyl)-5-isopropyl-biguanide hydrochloride (Proguanil hydrochloride) which comprises the steps of;
a) reacting p-chlorophenylcyanoguanidine (IV) with molar excess of isopropylamine (V) in suitable solvent in presence of metal salt;
b) adding acid to the obtained Proguanil metal complex;
c) adding chelating agent to the obtained reaction mass;
d) isolating Proguanil hydrochloride;
e) optionally purifying Proguanil hydrochloride to get pure compound of Formula I.

The compound of formula (V) is used in molar excess with respect to p-chlorophenylcyanoguanidine (IV), more preferably 4-5 equivalent.

The metal salt used is selected from copper oxide, copper sulfate, copper chloride preferably copper sulfate in the range of 0.5 mole to 3.0 mole with respect to cyanoguanidine, preferably 0.68 mole.

The suitable solvent is selected from tetrahydrofuran (THF), diglyme, ethoxyethanol, 1,4-dioxane, water or mixture thereof, preferably, THF-water.

The solvent is used in the range of 1-30 parts with respect to p-chlorophenylcyanoguanidine (IV), more preferably 6.0 parts and water is used in the range of 1-30 parts with respect to p-chlorophenylcyanoguanidine (IV), more preferably 5.0 parts.

The reaction is carried out at temperature 50-100° C., preferably 60-65° C. for 2-10 hours. Absence of p-chlorophenylcyanoguanidine (IV) is confirmed by checking the reaction mixture by thin layer chromatography (TLC). The organic solvent is recovered by distillation at 70-75° C. after completion of the reaction.

Acid used is hydrochloric acid in the range of 2-30 parts with respect to p-chlorophenylcyanoguanidine (IV), more preferably 2.5 parts and addition of acid is carried out at temperature range of 25-30° C.

The Proguanil copper complex is broken by adding chelating agent such as sodium sulphide or ethylene diamine tetraceticacid disodium salt (EDTANa₂) or nitrilotriacetic acid at temperature range of 5-25° C., preferably between 15-20° C. After complete addition the pH of the reaction mixture is in the range of 7.0-8.5, preferably 8.0.

Sodium sulfide is used in the range of 0.5 mole to 3.0 mole with respect to cyanoguanidine preferably 0.68 mole.

EDTANa₂ is used in the range of 0.5 to 5.0 moles, preferably 1.0 mole equivalent with respect to cyanoguanidine, preferably in presence of base like ammonia, methylamine or ethylamine preferably ammonia, more preferably 20% of ammoniacal EDTANa₂ solution is used.

According to another embodiment of the present invention there is provided process for purification of Proguanil hydrochloride comprising treating Proguanil hydrochloride, with suitable solvent to get pure Proguanil hydrochloride with purity more than 99%.

According to another embodiment of the present invention there is provided process for purification of Proguanil hydrochloride comprising the step of,
a) dissolving Proguanil hydrochloride in suitable solvent;
b) adding anti-solvent to the obtained solution to get pure Proguanil hydrochloride with purity more than 99%.

Suitable solvent used is selected from the group consisting of water, acetic acid, alcohol, toluene, ethyl acetate or mixtures thereof.

The alcohol may be selected from the group consisting of ethanol, methanol, isopropanol, n-propanol or mixtures thereof.

Anti-solvent used is selected from the group consisting of hydrocarbons, esters, ethers or mixtures thereof.

Said hydrocarbon is selected from petroleum ether, toluene, methylene chloride or ethylene chloride. Said ester is selected from methyl acetate or ethyl acetate.

Said ether is selected from methyl tert-butyl ether or diisopropylether.

Hereinafter, the present invention will be described in detail.

The preferred embodiment of the present invention provides reaction of p-chlorophenyl cyanoguanidine in suitable solvent with molar excess of isopropyl amine at 60-65° C. in presence of copper sulfate pentahydrate. The reaction mixture is refluxed for 2-10 hours. TLC was checked for the absence of p-chlorophenyl cyanoguanidine. Water is added to the obtained reaction mixture and solvent is distilled out at temperature 70-75° C. The organic solvent from the reaction mixture is distilled in the range of 1-7 parts with respect to p-chlorophenylcyanoguanidine (IV), more preferably 5.5 parts. Then the reaction mass is cooled to temperature 25-30° C. Acid solution is added to the cooled reaction mixture maintaining temperature between 25-30° C. and stirred for 30 minutes at the same temperature. Ammoniacal EDTA solution, prepared by mixing water, aqueous ammonia (25%) and EDTA disodium salt in a sufficient quantity, is added to the reaction mixture at temperature 15-20° C. The reaction mass is stirred at same temperature, the separated product is filtered, washed with cold water to wash out blue color and dried at temperature 90-95° C. to get Proguanil hydrochloride in high yield.

Proguanil hydrochloride thus obtained is purified by dissolving in water at 85-95° C., adding activated charcoal and stirring for 15 min. The hot mass is filtered over hyflobed and filtrate is stirred at temperature 10-15° C. to crystallize out the product. The product thus obtained is dissolved in suitable solvent at temperature 60-65° C., filtered and anti solvent is added to the stirred filtrate followed by cooling at temperature 10-15° C. to crystallize out pure Proguanil hydrochloride with purity more than 99% by HPLC preferably 99.9%.

In another preferred embodiment of the present invention, p-chlorophenyl cyanoguanidine is stirred in suitable solvent and copper sulfate pentahydrate and isopropylamine is added to the stirred solution. The reaction mixture is refluxed for 2-10 hours. TLC is checked for the absence of p-chlorophenyl cyanoguanidine. To the obtained reaction mixture, water is added and solvent is distilled out and recovered. The reaction mass is then cooled to temperature 25-30° C. and acid solution is added to the reaction mixture maintaining the same temperature followed by stirring for 30 minutes. The aqueous sodium sulfide solution is added to reaction mass at temperature 25-30° C. with proper scrubbing of hydrogen sulfide gas, stirred for 30 min. and filtered. The insoluble copper sulfide is filtered off and filtrate obtained is cooled at temperature 10-15° C. and 25% aqeuous ammonia is added maintaining the same temperature. The product obtained is filtered and dried at 90-95° C. with high yield of 75-80%.

Proguanil hydrochloride thus obtained is dissolved in water at 85-95° C. The hot mass is filtered over hyflobed and filtrate is stirred at 10-15° C. for 1 hour to get crystallized product which is further dissolved in organic solvent and the pure compound is re-precipitated by addition of anti-solvent. Proguanil hydrochloride (Formula-I) thus obtained has purity more than 99% (by HPLC), preferably 99.9%.

Alternatively Proguanil hydrochloride is dissolved in suitable solvent and isolating the separated product with purity more than 94.6% (by HPLC).

The term “molar excess” as used herein refers to the meaning as understood by the person skilled in the art.

Proguanil hydrochloride obtained by the process of the present invention has the following particle size distribution (PSD) data,
1) d(0.9) less than or equal to about 100μ,
2) d(0.5) less than or equal to about 50μ,
3) d(0.1) less than or equal to about 20μ.

The particles may be further micronized by techniques which are known in the art.

The present invention also provides a pharmaceutical formulation comprising Proguanil hydrochloride prepared by process of the present invention alone or in combination with other antimalarial agents in association with one or more pharmaceutically acceptable carriers.

Proguanil hydrochloride obtained by the process of the present invention exhibits the following XRPD,

| Pos. [°2Th] | Rel. Int. [%] |
|---|---|
| 7.14 | 100 |
| 9.95 | 1.88 |
| 14.12 | 28.50 |
| 14.83 | 7.51 |
| 15.67 | 2.73 |
| 19.30 | 8.02 |
| 19.87 | 22.78 |
| 21.41 | 4.22 |
| 21.98 | 30.02 |
| 22.75 | 5.28 |
| 23.87 | 31.30 |
| 24.80 | 7.24 |
| 25.38 | 46.83 |
| 25.71 | 20.29 |
| 28.26 | 13.26 |
| 29.74 | 8.12 |
| 30.69 | 5.60 |
| 32.45 | 5081 |
| 32.97 | 3.42 |
| 34.07 | 23.89 |
| 35.49 | 15.00 |
| 38.26 | 7.70 |
| 39.51 | 8.67 |
| 43.40 | 2.28 |
| 49.09 | 2.67 |

While the present invention has been described in terms of its specific embodiments, certain modifications and equivalents will be apparent to those skilled in the art and are included within the scope of present invention. The examples are provided to illustrate particular aspects of the disclosure and do not limit the scope of the present invention.

### Example: 1

### Reference Example

### Preparation of Proguanil Base

20 g (0.102 mole) p-chlorophenyl cyanoguanidine was stirred in 150 ml ethanol and 60 ml water at 25-35° C. 13.0 g (0.05 mole) copper sulfate pentahydrate was added to this stirring solution. 30 ml (0.34 mole) isopropylamine was added to this stirring solution. The reaction mixture was heated to reflux and maintained for 16 hrs. TLC was checked to confirm the absence of p-chlorophenyl cyanoguanidine. 500 ml water was added to the reaction mixture. Aqueous HCl solution (50 ml conc. HCl in 300 ml water) was added to reaction mixture maintaining temperature 25-30° C. and the reaction mass was stirred for 30 minutes. Then aqueous sodium sulfide solution (40 g sodium sulfide flakes the above reaction mass at 25-30° C. with proper scrubbing of hydrogen sulfide gas. After complete addition the reaction mixture was stirred for 30 min and the insoluble copper sulfide was filtered off. The obtained filtrate was cooled at 10-15° C. and aqueous sodium hydroxide solution (30 g sodium hydroxide in 300 ml water) was added dropwise at 10-15° C. followed by stirring. The separated solid product was filtered and dried at 90-95° C. to get 17 gm of 1-(4-chlorophenyl)-5-(1-methylethyl) biguanide (Proguanil base) with 80% purity (by HPLC).

### Example 2

### Preparation of Proguanil Hydrochloride

200 g (1.02 mole) p-chlorophenyl cyanoguanidine was stirred in 1200 ml tetrahydrofuran (THF) and 1000 ml water at 25-35° C. 170 g (0.68 mole) copper sulfate pentahydrate and 360 ml (4.14 mole) isopropylamine was added to this stirred solution. The reaction mixture was heated to reflux for 3 hours. TLC was checked for the absence of p-chlorophenyl cyanoguanidine. 800 ml water was added to the refluxed reaction mixture and THF was distilled out at 70-75° C. Then reaction mass was cooled to temperature 25-30° C. Aqueous HCl solution [500 ml conc. HCl in 800 ml water] was added to cooled reaction mixture and stirred for 30 minutes. Cooled ammoniacal EDTA solution [800 ml water, 360 ml aqueous ammonia (25%) and 352 g EDTA disodium salt] was added dropwise to the above reaction mixture maintaining the temperature 15-20° C. After complete addition the reaction mass was stirred at same temperature for 30 min and separated product was filtered, washed with cold 240 g of Proguanil hydrochloride.

[Yield: 79%]

### Example 3

### Purification of Proguanil Hydrochloride

235 g Proguanil hydrochloride obtained in example 2 was dissolved in 6.6 litre purified water at temperature 85-95° C. 12 g activated charcoal was added to it and stirred for 15 min. The hot mass was filtered over hyflobed and filtrate was stirred at 10-15° C. for 1 hour. The crystallized product was filtered and dried at 90-95° C. The solid material (152 g) obtained was dissolved in 760 ml methanol at 60-65° C. The solution was filtered and 3.8 litre ethyl acetate was added to the filtrate followed by stirring and cooling at 10-15° C. The crystallized product was filtered, washed with 150 ml cold ethyl acetate and dried at 70-75° C. 125 g pure Proguanil hydrochloride was obtained. [Purity: 99.9% by HPLC]

### Example: 4

### Preparation of Proguanil Hydrochloride

200 g (1.02 mole) p-chlorophenyl cyanoguanidine was stirred in 1200 ml THF and 1000 ml water at 25-35° C. 170 g (0.68 mole) copper sulfate pentahydrate and 360 ml (4.14 mole) isopropylamine was added to this stirring solution. The reaction mixture was heated to reflux for 3 hours and TLC was checked for the absence of p-chlorophenyl cyanoguanidine. 800 ml water was added to the obtained reaction mixture and THF was distilled out at temperature 70-75° C. solution [500 ml conc. HCl in 800 ml water] was added to the cooled reaction mixture at the same temperature and stirred for 30 minutes. Aqueous sodium sulfide solution [106 g sodium sulfide flakes (assay 50%) (0.68 M) in 440 ml water] was added dropwise to the above obtained reaction mass at 25-30° C. with proper scrubbing of hydrogen sulfide gas. After complete addition the reaction mass was stirred for 30 min. and the insoluble copper sulfide was filtered off and filtrate obtained was cooled at temperature 10-15° C. Aqueous ammonia solution [360 ml (25%) aqueous ammonia and 800 ml water] was added dropwise to the cooled reaction mixture at temperature 10-15° C. followed by stirring. The obtained product was filtered and dried at 90-95° C. to get 240 g of Proguanil hydrochloride.

[Yield: 79%]

### Example: 5

### Purification of Proguanil hydrochloride

235 g Proguanil hydrochloride obtained in example 4 was dissolved in 6.6 litre purified water at 85-95° C. The hot mass was filtered over hyflobed and filtrate was stirred at 10-15° C. for 1 h. The crystallized product was filtered and dried at 90-95° C. The material (152 g) obtained was dissolved in 760 ml methanol at temperature 60-65° C. and hot mass was filtered. 3.8 litre ethyl acetate was added to the filtrate, stirred and cooled to 10-15° C. The crystallized product was filtered, washed with 150 ml cold (cooled at temperature 15° C.) ethyl acetate and dried at 70-75° C. 125 g pure Proguanil hydrochloride was obtained. [Purity 99.7% by HPLC]

### Example 6

### Preparation of Proguanil Hydrochloride

10 g (0.05 mole) p-chlorophenyl cyanoguanidine was stirred in 100 ml methanol and 50 ml water at 25-35° C. 6.5 g (0.03 mole) copper sulfate pentahydrate was added to this stirring solution. 15 ml (0.15 mole) isopropylamine was added to this stirring solution. The reaction mixture was refluxed for 3 hours. TLC was checked to confirm the absence of p-chlorophenyl cyanoguanidine. 40 ml water was added to the refluxed reaction mixture and methanol was distilled out at temperature 70-75° C. Then the reaction mass was cooled to 25-30° C. Aqueous HCl solution [25 ml conc. HCl in 80 ml water] was added to reaction mixture at 25-30° C., stirred for 30 minutes and sodium sulfide solution [4 g sodium sulfide dissolved in 16 ml water], cooled to temperature 25-30° C., was added dropwise to above reaction mixture. After complete addition, the reaction mass was stirred at same temperature for 30 min and the separated copper sulfide was filtered. Filtrate was cooled to 15-20° C. and 25 ml of 25% ammonia was added. The separated solid was filtered, washed and dried at 90-95° C. to get 7 g of Proguanil hydrochloride. Purity: 81% (by HPLC).

### Example 7

### Preparation of Proguanil Hydrochloride

5 g (0.025 mole) p-chlorophenyl cyanoguanidine was stirred in 50 ml water at 25-35° C. 4.25 g (0.017 mole) copper sulfate pentahydrate was added to this stirring solution. 9 ml (0.12 mole) isopropylamine was added to this stirring for 3 hours. TLC was checked after 3 hours. But there was no formation of the product. So reaction was continued for further 4 hours still the TLC did not showed the formation of the product this indicates that no reaction takes place when water alone is used as solvent system.

### Example 8

### Preparation of Proguanil Hydrochloride

20 g (0.10 mole) p-chlorophenyl cyanoguanidine was stirred in 200 ml ethanol and 100 ml water at 25-35° C. 12.5 g (0.05 mole) copper sulfate pentahydrate was added to this stirring solution. 30 ml (0.4 mole) isopropylamine was added to this stirring solution. The reaction mixture was heated to reflux for 3 hours. TLC was checked which shows presence of p-chlorophenyl cyanoguanidine. The reaction was continued further for 4 more hours and TLC was checked, which still showed presence of p-chlorophenyl cyanoguanidine. That means 0.5 mole of copper sulfate pentahydrate with respect to p-chlorophenyl cyanoguanidine is not sufficient to consume the entire amount of p-chlorophenyl cyanoguanidine.

### Example 9

### Preparation of Proguanil Hydrochloride

200 g (1.02 mole) p-chlorophenyl cyanoguanidine was stirred in 1200 ml THF and 1000 ml water at 25-35° C. 170 g (0.68 mole) copper sulfate pentahydrate was added to this stirring solution. 340 ml (4 mole) isopropylamine was added to this stirring solution. The reaction mixture was heated to reflux for 3 hours. TLC was checked to confirm the absence of p-chlorophenyl cyanoguanidine. 800 ml water was added to the above reaction mixture and THF was distilled out at 70-75° C. (around 1100-1200 ml solvent recovered). Then reaction mass was cooled to 25-30° C. and aqueous HCl solution [500 ml conc. HCl in 800 ml water] was added to reaction mixture at the same temperature and stirred for 30 minutes. Aqueous sodium sulfide solution [106 g sodium sulfide flakes (assay 50%, 0.68 mole) in 440 ml water] was added dropwise to the stirred reaction mass at 25-30° C. with proper scrubbing of hydrogen sulfide gas. After complete addition the reaction mass was stirred for 30 min and the insoluble copper sulfide was filtered off. The obtained filtrate was cooled at 10-15° C. and aqueous ammonia solution (360 ml of 25% aqueous ammonia diluted with 800 ml water) was added dropwise at 10-15° C. The reaction mixture was stirred, product was filtered and dried at 90-95° C. to get 240 g of Proguanil hydrochloride. [Yield: 79%]

### Example 10

### Purification of Proguanil Hydrochloride

5 g Proguanil hydrochloride was dissolved in 20 ml toluene at 100-110° C. Hot mass was filtered through hyflobed and the obtained filtrate was cooled under stirring. The separated product was filtered, washed with toluene and dried to get 3 g of crystallized product.

Purity: 96.3% by HPLC.

### Example 11

### Purification of Proguanil Hydrochloride

5 g Proguanil hydrochloride was dissolved in 12 ml ethyl acetate at 25-30° C. product was filtered, washed and dried to get 4.5 g crystallized material.

Purity: 94.6% by HPLC

### Example 12

### Purification of Proguanil Hydrochloride

8 g Proguanil hydrochloride was dissolved in 160 ml water at temperature 90-95° C. 0.5 g activated charcoal was added to it and stirred at 90° C. for 1 h. The obtained hot mass was filtered over hyflobed and filtrate was cooled under stirring. The separated product was filtered, washed with water and dried to get 5 g crystallized material.

Purity: 96% by HPLC

### Example: 13

### Purification of Proguanil Hydrochloride

8 g Proguanil hydrochloride was dissolved in 20 ml ethanol and 80 ml water at 80-85° C. 0.5 g activated charcoal was added to it and stirred at 90° C. for 1 hour. The obtained hot mass was filtered over hyflobed and filtrate was cooled under stirring. Separated product was filtered, washed with water and dried to get 5 g crystallized material.

Purity: 95.98% by HPLC

### Example: 14

### Purification of Proguanil Hydrochloride

8 g Proguanil hydrochloride was dissolved in 160 ml acetic acid at 80-90° C. 0.5 g activated charcoal was added to it and stirred at 90° C. for 1 hour. The obtained hot mass was filtered over hyflobed and filtrate was cooled under stirring. Separated product was filtered, washed with water and dried to get 4.5 g crystallized material.

Purity: 98.98% by HPLC

### Example 15

### Preparation of Proguanil

19.5 g (0.10 mole) p-chlorophenyl cyanoguanidine was stirred in 140 ml ethanol and 8.9 gm (0.15 mole) isopropylamine were stirred at 25-35° C. 6.8 gm (0.049 mole) of zinc chloride was dissolved in 25 ml of water and this solution was added to the obtained reaction mixture. The reaction mass was refluxed for 20 hrs. After completion of reaction the solvent was distilled out. 20 gm sodium hydroxide was dissolved in 250 ml water to make 8% sodium hydroxide solution and added to the obtained residue of reaction mass followed by stirring for 30 minutes. The undissolved solid was filtered, washed and suck dried. Purity: 30.14%

Total impurities: 59.87%, Sulfated ash: 22.8%

We claim:

1. A process for preparation of 1-(4-chlorophenyl)-5-isopropyl-biguanide or salts thereof comprising reaction of p-chlorophenylcyanoguanidine (IV) with molar excess of isopropylamine (V) and optionally converting into its salts

2. A process for preparation of 1-(4-chlorophenyl)-5-isopropyl-biguanide hydrochloride (Proguanil hydrochloride) which comprises the steps of:
a) reacting p-chlorophenylcyanoguanidine (IV) with molar excess of isopropylamine (V) in suitable solvent in presence of metal salt; 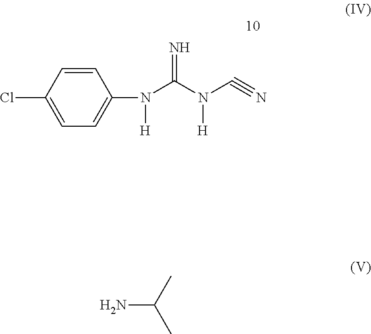
b) adding acid to the obtained Proguanil metal complex;
c) adding chelating agent to the obtained reaction mass;
d) isolating Proguanil hydrochloride;
f) optionally purifying Proguanil hydrochloride to get pure compound of formula I

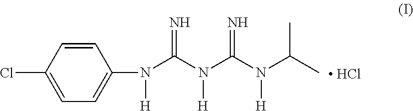

3. The process as claimed in claim 2 wherein said suitable solvent used is selected from tetrahydrofuran (THF), diglyme, ethoxyethanol, 1,4-dioxane, water or mixture thereof

4. The process as claimed in claim 2 wherein said suitable solvent is THF-water

5. The process as claimed in claim 2 wherein said metal salt is selected from copper oxide, copper sulfate, copper chloride and used in the range of 0.5 mole to 3.0 mole with respect to cyanoguanidine

6. The process as claimed in claim 2 wherein the reaction is performed at temperature of 50-100° C.

7. The process as claimed in claim 2 wherein acid used is hydrochloric acid

8. The process as claimed in claim 2 wherein said chelating agent used is selected from sodium sulfide or ethylene diamine tetraceticacid disodium salt (EDTANa₂) or nitrilotriacetic acid optionally in presence of base

9. The process as claimed in claim 8 wherein sodium sulfide is used in the range of 0.5 mole to 3.0 mole with respect to cyanoguanidine

10. The process as claimed in claim 8 wherein EDTANa₂ is used in the range of 0.5 to 5.0 moles equivalent with respect to cyanoguanidine

11. The process as claimed in claim 8 wherein said base used is selected from ammonia, methylamine or ethylamine

12. The process as claimed in claim 2 wherein the isolation step comprises dissolving Proguanil hydrochloride in water at 85-95° C. and stirring the solution at 10-15° C. to get crystallized product

13. A process for purification of Proguanil hydrochloride comprising the step of,
a) dissolving Proguanil hydrochloride in suitable solvent;
b) isolating pure Proguanil hydrochloride

14. A process for purification of Proguanil hydrochloride comprising the step of,
a) dissolving Proguanil hydrochloride in suitable solvent;
b) adding anti-solvent to the obtained solution of step a) to get pure Proguanil hydrochloride with purity more than 99%

15. The process as claimed in claim 13 wherein suitable solvent used is selected from the group consisting of water, acetic acid, ethanol, methanol, isopropanol, propanol or mixtures thereof

16. The process as claimed in claim 14 wherein said anti-solvent is selected from the group consisting of hydrocarbons such as petroleum ether, toluene, methylene chloride and ethylene chloride, esters such as methyl acetate, ethyl acetate or ethers such as methyl tert-butyl ether, diisopropylether or mixtures thereof, preferably ethyl acetate

17. The process as claimed in claim 1 wherein Proguanil hydrochloride obtained has particle size distribution (PSD) of d(0.9) less than or equal to about 100μ, d(0.5) less than or equal to about 50μ and d(0.1) less than or equal to about 20μ

18. Proguanil hydrochloride characterized by X-ray diffraction pattern having peaks at about 2 θ:7.14, 9.97, 14.12, 14.83, 15.67, 19.30, 19.87, 21.41, 21.98, 22.75, 23.87, 24.80, 25.38, 25.71±0.2 deg

19. The process as claimed in claim 14 wherein suitable solvent used is selected from the group consisting of water, acetic acid, ethanol, methanol, isopropanol, propanol or mixtures thereof
